(19) United States
(12) Patent Application Publication
(10) Pub. No.: US 2015/0150947 A1
(43) Pub. Date: Jun. 4, 2015

(54) TREATING INFLAMMATION USING SERELAXIN

(71) Applicant: Elaine Unemori, Oakland, CA (US)

(72) Inventor: Elaine Unemori, Oakland, CA (US)

(21) Appl. No.: 14/418,107

(22) PCT Filed: Jul. 29, 2013
(86) PCT No.: PCT/US2013/052536

§ 371 Date: Jan. 29, 2015

Related U.S. Application Data
(60) Provisional application No. 61/677,688, filed on Jul. 31, 2012.

(51) Int. Cl. A61K 38/22

(52) U.S. Cl. CPC A61K 38/2221

(57) ABSTRACT

The present disclosure relates to methods of treating inflammation in a subject. Particularly, the disclosure provides methods for treating inflammation by administering pharmaceutically active serelaxin in order to increase a soluble marker associated with reducing inflammation. Further encompassed in the present disclosure are method for treating inflammatory disorders and kits for administering pharmaceutically active serelaxin to subjects suffering from such disorders.

### FIELD

The present disclosure relates to methods of treating inflammation in a subject. Particularly, the disclosure provides methods for treating inflammation by administering pharmaceutically active serelaxin in order to increase a soluble marker associated with reducing inflammation. Further encompassed in the present disclosure are methods for treating inflammatory disorders and kits for administering pharmaceutically active serelaxin to subjects suffering from such disorders.

### BACKGROUND

Inflammation

Inflammation is generally understood to be the immune system's response to protect the body from invaders and infection. When inflammation occurs, white blood cells move out of the blood and into the affected area of the body where they act as phagocytes, i.e., they destroy foreign pathogens. Inflammation includes swelling at the site of invasion or infection, which in turn compresses nerve endings resulting in pain. Symptoms of inflammation include fatigue, loss of energy, headaches, fever and chills. In some instances, the inflammatory response can be triggered even though there is no foreign invader or pathogen as, for example, in autoimmune disorders such as rheumatoid arthritis. In autoimmune disorders, the body's immune system attacks its own tissues and organs. In addition, inflammation is now known to be a by-product of many diseases including atherosclerosis, heart disease, Alzheimer's disease (ALZ) and cancer. Excessive inflammation is a major contributor to disease progression.

Interleukin-33

The Interleukin-1 (IL-1) family includes a group of cytokines that play an important role in the regulation of inflammatory responses. Interleukin-33 (IL-33) is a newly identified member of this family and is expressed in many cell types following proinflammatory stimulation. IL-33 functions as the ligand for the receptor ST-2, which is widely expressed on T helper 2 (TH2) cells and mast cells. IL-33 appears to have a dual role in disease progression, including protecting the host against helminth infection and reducing atherosclerosis by promoting TH2 immune responses. However, IL-33 can also exacerbate the pathogenesis of TH2 and mast cell mediated inflammatory diseases including asthma, joint inflammation, atopic dermatitis, and anaphylaxis. Thus, IL-33 is a promising new target for therapeutic intervention (see Liew et al. (2012) Nature Reviews 10:103-110).

While numerous anti-inflammatory treatments are available, they often are ineffective or cause unwanted side effects, e.g., immune suppression. There remains a strong unmet need for effective anti-inflammatory treatments that do not cause unwanted side effects and that target the IL-33 anti-inflammatory pathway.

### BRIEF SUMMARY

The disclosure provides a method of treating disorders resulting from inflammation. One aspect of the disclosure provides a method of treating disorders resulting from or associated with IL-33 mediated inflammation. Particularly, the disclosure provides a method of treating inflammation, including administering to a subject a pharmaceutically active serelaxin in a dose effective to cause transient up-regulation of soluble ST-2 (sST-2) in a tissue of the subject affected by the inflammation, wherein the transient up-regulation of sST-2 reduces proinflammatory cytokines in the tissue. Serelaxin transiently upregulates sST-2, thus reducing inflammation by lowering the level of proinflammatory cytokines. IL-33 binds the membrane bound form of the ST-2 receptor, thus potently inducing proinflammatory cytokines. The soluble form of the ST-2 receptor is a decoy that also binds to IL-33 but does not induce proinflammatory cytokines.

Another aspect of the disclosure provides a method of treating IL-33 mediated inflammation, wherein pharmaceutically active serelaxin is administered such that sST-2 is transiently up-regulated in a specific tissue, including but not limited to, lung tissue, skin tissue, joint tissue, nerve tissue and/or vascular tissue. The dose of pharmaceutically active serelaxin ranges from about 10 μg/kg/day to about 500 μg/kg/day, and more specifically, from about 30 μg/kg/day to about 250 μg/kg/day. The inflammation that can be treated with serelaxin includes inflammatory disorders that are mediated by IL-33. Such disorders include, but are not limited to, eosinophilic airway hyperresponsiveness, asthma, rheumatoid arthritis, multiple sclerosis (MS), ankylosing spondylitis (AS), inflammatory bowel disease, gout, myositis, Sjögren's syndrome, systemic lupus erythematosus (SLE), vasculitis, pleural malignancy, sepsis, trauma, wound healing, atopic allergy, anaphylaxis, autoimmune encephalomyelitis, CNS hypoxia, CNS vascular damage, and hypernociception. In addition, serelaxin can be used to treat inflammatory skin disorders that are mediated by IL-33, including but not limited to, eczema, dermatitis, scleroderma, poison ivy, acne, hives and psoriasis. The inflammation that can be treated with serelaxin further includes an inflammatory disorder that is an IL-33-mediated autoimmune disorder or is based on an IL-33 mediated immune response.

Yet, another aspect of the disclosure provides a method of treating IL-33 mediated inflammation, wherein pharmaceutically active serelaxin is administered such that sST-2 is transiently up-regulated in a specific tissue, wherein the transient up-regulation of sST-2 lasts from about 1 day to about 5 days, and more specifically, from about 2 days to about 4 days. In one embodiment, sST-2 functions as a decoy receptor and binds IL-33. As a decoy receptor, sST-2 lacks a transmembrane and cytoplasmic domain and simply functions to capture or trap IL-33 to keep it from binding to its natural receptor ST-2. Thus, sST-2 inhibits binding of IL-33 to its transmembrane receptor ST-2.

The disclosure further encompasses a test kit that includes serelaxin in a dose effective to cause transient up-regulation of sST-2 in a tissue of a subject affected by inflammation; and instructions describing a pharmaceutical treatment regimen based on the transient up-regulation as compared to an established treatment model. Herein, the dose of serelaxin ranges from about 10 μg/kg/day to about 500 μg/kg/day and the pharmaceutical treatment regimen is tissue specific.

The disclosure further contemplates a method of treating inflammation, including administering to a subject a pharmaceutically active serelaxin in a dose effective to cause transient up-regulation of sST-2 in a tissue of the subject affected by inflammation, and determining if the transient up-regulation of soluble ST-2 reduced proinflammatory cytokines in the tissue of the subject. The reduced proinflammatory cytokines are evaluated by using an assay that measures serum levels of the proinflammatory cytokines in the peripheral blood of the subject. An example of such an assay is an ELISA assay. Examples of reduced proinflammatory cytokines include, but are not limited to, IL-1, IL-3, IL-5, IL-6, IL-13, IL-33, TNF, CXCL2, CCL2, CCL3, CCLS, CCL17, CCL24, PGD2 and LTB4.

### BRIEF DESCRIPTION OF THE DRAWINGS

The present disclosure is best understood when read in conjunction with the accompanying figures, which illustrate the invention. It is understood, however, that the disclosure is not limited to the specific embodiments disclosed in the figures.

FIG. 1 shows a graph that depicts the change from baseline in median sST-2 concentrations with time in subjects treated for 48 hours with 4 doses of serelaxin and placebo. Whereas sST-2 declines in the placebo group at 48 hours, the serelaxin groups show a transient increase relative to placebo in sST-2 levels at 48 hours. The difference between the placebo group and each of the serelaxin dose groups is statistically significant at 48 hours.

FIG. 2 depicts the change from baseline in median sST-2 concentrations with time in subjects in the placebo group and the pooled serelaxin group.

FIG. 3 illustrates a between-treatment analysis of sST-2 by visit, comparing a change from baseline in sST-2 geometric means between the serelaxin groups compared to the placebo group at 3 time points. At 48 hours of treatment, change from baseline in all serelaxin groups, as well as the pooled serelaxin group, was significantly different from the change from baseline in the placebo group.

### DETAILED DESCRIPTION OF THE EMBODIMENTS

### General Overview

The present disclosure relates to methods of treating disorders associated with inflammation by administering serelaxin to subjects who suffer from such inflammation and related disorders. Particularly, these methods include administering pharmaceutically active serelaxin in order to cause up-regulation of soluble ST-2 (sST-2) in a tissue of a subject that is affected by IL-33 mediated inflammation. The up-regulation of sST-2 reduces proinflammatory cytokines in the tissue and thereby reduces inflammation in a tissue specific matter. For example, IL-33 mediated inflammation is a major contributor to many diseases and disorders, it has been associated with the steady decline of a subject's health. Proinflammatory cytokines include, but are not limited to, interleukin-1-beta (IL-1), interleukin-3 (IL-3), interleukin-5 (IL-5), interleukin-6 (IL-6), interleukin-13 (IL-13), tumor necrosis factor (TNF), CXC-chemokine ligand 2 (CXCL2), CC-chemokine ligand 2 (CCL2), CC-chemokine ligand 3 (CCL3), CC-chemokine ligand 5 (CCLS), CC-chemokine ligand 17 (CCL17), CC-chemokine ligand 24 (CCL24), prostaglandin D2 (PGD2) and leukotriene B4 (LTB4) as well as IL-33. Numerous inflammatory disorders have been associated with IL-33 mediated inflammation, including, but not limited to, rheumatoid arthritis, multiple sclerosis (MS), ankylosing spondylitis (AS), inflammatory bowel disease, asthma, gout, sepsis, anaphylaxis, autoimmune encephalomyelitis, CNS hypoxia, CNS vascular damage, hypernociception, eczema, dermatitis, scleroderma, poison ivy, acne, hives, and psoriasis. Furthermore, chronic inflammation that is mediated by IL-33 is a common side effect of diseases that are not traditionally known as inflammatory disorders such as cancer. Treatments for inflammation are available, however, many of the existing anti-inflammatory medications have side effects such as nausea, vomiting, diarrhea, constipation, rash, dizziness, headache, drowsiness, and edema. The more severe side effects include kidney failure, liver failure, ulcer, prolonged bleeding after an injury, stroke and heart attack, which can put a subject at serious risk. In addition, existing treatments are not always effective. Hence, subjects who are afflicted with inflammation are in need of new therapeutic methods that improve the condition and stabilize the subjects without causing these side effects. Serelaxin is a naturally occurring substance that, when administered to a subject with inflammation, can reduce the proinflammatory cytokines in the tissue that are associated with IL-33 mediated inflammation without affecting the neighboring tissue and without causing noticeable side effects. Since serelaxin works by transiently increasing a biochemical substance (i.e., a soluble marker), which in turn down-regulates proinflammatory cytokines, the inflammation is reduced when needed but the inflammatory response system is not affected to such an extent that serious side affects occur. As such, serelaxin offers a new form of treatment for IL-33 mediated inflammation, which is specifically targeted to a particular tissue and transient enough to not interrupt the body's natural defense system.

Without wanting to be bound by theory, serelaxin is contemplated to affect the ST-2 pathway by stimulating a decoy receptor or soluble ST-2 receptor (sST-2) to bind to an inducer of one or more proinflammatory cytokines. In one embodiment, the decoy receptor binds an excess of ligand, thereby reducing or modulating the amount of ligand that binds to its natural receptor. In another embodiment, the decoy receptor binds most or all of the ligand, thereby inhibiting all or most of the ligand that normally binds to its natural receptor.

For example, IL-33 is a major inducer of proinflammatory cytokines and the capture of this ligand by its decoy receptor reduces and modulates inflammation in the tissue. The effect of serelaxin on the decoy is transient, and thus, it can ameliorate inflammation by inhibiting excess IL-33 and breaking the cycle of progression while not adversely affecting tissues believed to potentially benefit from the effects of IL-33. One of the unwanted consequences of attempting to treat IL-33-mediated inflammatory disorders by administering sST-2 directly is that a purported cardiovascular protective effect of IL-33 could be impacted, thereby increasing the potential for increased risk of cardiovascular morbidity. While elevated sST-2 has been reported to be a predictor of poor outcome in subjects with AHF, serelaxin acts as a vasomodulator and has favorable hemodynamic effects, greatly reducing a risk of adverse cardiac events. As such, serelaxin is contemplated to be capable of modulating the inflammatory response while simultaneously modulating positive systemic and renal hemodynamic function in these subjects. In addition, it has been shown in animal models that IL-33 reduces cardiac hypertrophy and fibrosis and that sST-2, by virtue of its IL-33 inhibitory activity, exacerbates these effects. Direct administration of sST-2 could therefore favor these adverse cardiac effects. However, because serelaxin is a mediator of extracellular matrix degradation and has been shown to inhibit cardiac fibrosis, transient sST-2 induction by serelaxin is predicted to have no detrimental effects on cardiac fibrosis and hypertrophy.

### DEFINITIONS

The term “inflammation” includes, for the purpose of the specification and claims, an accumulation, up-regulation and/or induction of proinflammatory agents in a tissue and/or organ of a mammalian subject.

The term “serelaxin” refers to a peptide hormone that is identical to relaxin in its amino acid sequence. Serelaxin encompasses human serelaxin, including intact full-length human serelaxin or a portion of the serelaxin molecule that retains biological activity. The term “serelaxin” encompasses human H1 preproserelaxin, proserelaxin, and serelaxin; H2 preproserelaxin, proserelaxin, and serelaxin; and H3 preproserelaxin, proserelaxin, and serelaxin. The term “serelaxin” further includes biologically active (also referred to herein as “pharmaceutically active”) serelaxin from recombinant, synthetic or native sources as well as serelaxin variants, such as amino acid sequence variants. As such, the term contemplates synthetic human serelaxin and recombinant human serelaxin, including synthetic H1, H2 and H3 human serelaxin and recombinant H1, H2 and H3 human serelaxin. The term further encompasses active agents with serelaxin-like activity, such as serelaxin agonists and/or serelaxin analogs and portions thereof that retain biological activity, including all agents that competitively displace bound serelaxin from a serelaxin receptor (e.g., RXFP1 receptor, RXFP2 receptor, RXFP3 receptor, RXFP4 receptor, previously known as LGR7, LGR8, GPCR135, GPCR142, respectively). Thus, a pharmaceutically effective serelaxin or serelaxin agonist is any agent with serelaxin-like activity that is capable of binding to a serelaxin receptor to elicit a serelaxin-like response. In addition, a pharmaceutically effective serelaxin or serelaxin agonist is any agent with serelaxin-like activity that is capable of up-regulating and/or modifying the sST-2 decoy receptor activity, and further capable of down-regulating and/or modifying the IL-33 activity, thereby modulating and/or changing and/or decreasing the amount of proinflammatory cytokines that are present in a tissue and/or organ during and/or at the onset of inflammation. In addition, the nucleic acid sequence of serelaxin as used herein must not be 100% identical to nucleic acid sequence of human serelaxin (e.g., H1, H2 and/or H3) but may be at least about 40%, 50%, 60%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% identical to the nucleic acid sequence of human serelaxin. Serelaxin, as used herein, can be made by any method known to those skilled in the art. Examples of such methods are illustrated, for example, in U.S. Pat. No. 5,759,807 as well as in Bullesbach et al. (1991) The Journal of Biological Chemistry 266:10754-10761. Examples of serelaxin molecules and analogs are illustrated, for example, in U.S. Pat. No. 5,166,191. Naturally occurring biologically active serelaxin may be derived from human, murine (i.e., rat or mouse), porcine, or other mammalian sources. Also encompassed is serelaxin modified to increase in vivo half life, e.g., PEGylated serelaxin (i.e., serelaxin conjugated to a polyethylene glycol), modifications of amino acids in serelaxin that are subject to cleavage by degrading enzymes, and the like. The term also encompasses serelaxin comprising A and B chains having N- and/or C-terminal truncations. In general, in H2 serelaxin, the A chain can be varied from A(1-24) to A(10-24) and B chain from B(1-33) to B(10-22); and in H1 serelaxin, the A chain can be varied from A(1-24) to A(10-24) and B chain from B(1-32) to B(10-22). Also included within the scope of the term “serelaxin” are other insertions, substitutions, or deletions of one or more amino acid residues, glycosylation variants, unglycosylated serelaxin, organic and inorganic salts, covalently modified derivatives of serelaxin, preproserelaxin, and proserelaxin. Also encompassed in the term is a serelaxin analog having an amino acid sequence, which differs from a wild-type (e.g., naturally-occurring) sequence, including, but not limited to, serelaxin analogs disclosed in U.S. Pat. No. 5,811,395. Possible modifications to serelaxin amino acid residues include the acetylation, formylation or similar protection of free amino groups, including the N-terminal, amidation of C-terminal groups, or the formation of esters of hydroxyl or carboxylic groups, e.g., modification of the tryptophan (Trp) residue at B2 by addition of a formyl group. The formyl group is a typical example of a readily-removable protecting group. Other possible modifications include replacement of one or more of the natural amino-acids in the B and/or A chains with a different amino acid (including the D-form of a natural amino-acid), including, but not limited to, replacement of the Met moiety at B24 with norleucine (Nle), valine (Val), alanine (Ala), glycine (Gly), serine (Ser), or homoserine (HomoSer). Other possible modifications include the deletion of a natural amino acid from the chain or the addition of one or more extra amino acids to the chain. Additional modifications include amino acid substitutions at the B/C and C/A junctions of proserelaxin, which modifications facilitate cleavage of the C chain from proserelaxin; and variant serelaxin comprising a non-naturally occurring C peptide, e.g., as described in U.S. Pat. No. 5,759,807. Also encompassed by the term “serelaxin” are fusion polypeptides comprising serelaxin and a heterologous polypeptide. A heterologous polypeptide (e.g., a non-serelaxin polypeptide) fusion partner may be C-terminal or N-terminal to the serelaxin portion of the fusion protein. Heterologous polypeptides include immunologically detectable polypeptides (e.g., “epitope tags”); polypeptides capable of generating a detectable signal (e.g., green fluorescent protein, enzymes such as alkaline phosphatase, and others known in the art); therapeutic polypeptides, including, but not limited to, cytokines, chemokines, and growth factors. All such variations or alterations in the structure of the serelaxin molecule resulting in variants are included within the scope of this disclosure so long as the functional (biological) activity of the serelaxin is maintained. Preferably, any modification of the serelaxin amino acid sequence or structure is one that does not increase its immunogenicity in the individual being treated with the serelaxin variant. Those variants of serelaxin having the described functional activity can be readily identified using in vitro and in vivo assays known in the art.

The term “subject” refers to a mammal, including but not limited to, humans, primates, rodents, rabbits, marine mammals, hoofed mammals, and carnivores. The term “subject” further encompasses laboratory and research mammals, including experimental animals.

The term “administering” refers to giving or applying to a subject a pharmaceutical remedy or formulation via a specific route, including but not limited to, intravenously, subcutaneously, intramuscularly, sublingually, intranasally, intracerebrally, intracerebroventricularly, topically, intravitrealy and via inhalation.

The term “effective” as in “effective to cause transient up-regulation of soluble ST-2 in a tissue of a subject affected by inflammation” refers to the amount of pharmaceutically active serelaxin that will result in a measurable desired medical or clinical benefit to a subject suffering from inflammation, as compared to the untreated or placebo-treated subject (i.e., a subject suffering from inflammation that is not treated with serelaxin).

The term “up-regulation” refers to a positive regulatory effect on a physiological process at the molecular, cellular, or systemic level. For example, in one embodiment, it refers to a process by which a cell increases the quantity of a cellular component, such as RNA and/or protein by up-regulating gene expression.

The term “decoy receptor”, as used herein, generally refers to a receptor that binds a specific ligand, thereby preventing the ligand from binding to its natural receptor (e.g., transmembrane ST-2 receptor).

Serelaxin

Serelaxin is a polypeptide hormone that is similar in size and shape to insulin. It is an endocrine and autocrine/paracrine hormone belonging to the insulin gene superfamily. The active form of the encoded protein consists of an A chain and a B chain, held together by disulphide bonds, two inter-chains and one intra-chain. Thus, the structure closely resembles insulin in the disposition of disulphide bonds. In humans, there are three known non-allelic serelaxin genes, serelaxin-1 (RLN-1 or H1), serelaxin-2 (RLN-2 or H2) and serelaxin-3 (RLN-3 or H3). H1 and H2 share high sequence homology. There are two alternatively spliced transcript variants encoding different isoforms described for this gene. H1 and H2 are differentially expressed in reproductive organs (U.S. Pat. No. 5,023,321 and Garibay-Tupas et al. (2004) Molecular and Cellular Endocrinology 219:115-125), while H3 is found primarily in the brain. The evolution of the serelaxin peptide family in its receptors is generally well known in the art (Wilkinson et al. (2005) BMC Evolutionary Biology 5:1-17; and Wilkinson & Bathgate (2007), Chapter 1, Serelaxin and Related Peptides, Landes Bioscience and Springer Science+Business Media).

Serelaxin and the ST-2 Intracellular Signaling Pathway

ST-2 is a member of the Interleukin-1 (IL-1) family, which belongs to the Toll-like receptor (TLR/IL-1R (TIR)) superfamily. The gene for ST-2 is conserved across species. It spans approximately 40 kb on human chromosome 2q12 and is part of the larger human Interleukin-1 (IL-1) gene cluster (see Genbank accession number AC007248). In its transmembrane form, the ST-2 gene functions as a pro-inflammatory mediator, and in its soluble form it function as an anti-inflammatory inhibitor of T helper type 2 (TH2) function. Thus, there are two types of ST-2 receptors, i.e., the membrane bound isoform of ST-2 and the soluble isoform of ST-2 (sST-2). A ligand for ST-2 is Interleukin-33 (IL-33). However, sST-2 does not signal but functions as a decoy receptor for IL-33, thereby preventing its binding to the transmembrane receptor ST-2.

Serelaxin administration to subjects with AHF is associated with improvements in dyspnea and improved mid- and long term outcomes. Because sST-2 is predictive of poor outcomes in AHF, the expectation was that serelaxin administration would be associated with a decrease in sST-2 levels. However, contrary to this expectation, the inventor has found that, surprisingly, serelaxin induces the expression of sST-2. Yet, sST-2 is known to capture IL-33, preventing IL-33 from signaling. Thus, in a defined tissue- and IL-33-mediated specific way, this leads to a reduction in the recruitment of proinflammatory cytokines. This is a unprecedented finding because serelaxin has been associated with a pro-inflammatory response (Figueiredo et al. (2006) The Journal of Biological Chemistry 281:3030-3039; Bryant-Greenwood et al. (2009) Placenta 30:599-606; Horton et al. (2012) Placenta 33:399-407); an anti-inflammatory response (Masini et al. (2006) Free Radic. Biol. Med. 39:520-531; Cosen-Binker et al. (2006) World J. Gastroenterol. 12:1558-1568; Santora et al. (2007) J Pharmacol. Exp. Ther. 322:887-893; Brecht et al. (2011) Regul. Pept. 166:76-82); a pro-TH1 inflammatory response (Piccinni et al. (1999) Eur. J. Immunology 29:2241-2247); an anti-neutrophil response (Masini et al. (2004) Endocrinology 145:1106-1112); an absence of an effect on inflammation (Mookerjee et al. (2006) Endocrinology 147:754-761; Hewitson et al. (2007) Endocrinology 148:660-669; Samuel et al. (2007) Endocrinology 148:4259-4266; Royce et al. (2009) Endocrinology 150: 2692-2699); as well as a mixed inflammatory effect (Horton et al. (2011) Biol. Reprod. 85:788-797). The finding that serelaxin induces sST-2 was not expected in light of the existing literature. Notably, this finding allows for a new therapeutic approach by using serelaxin for the treatment of disorders that are related to aberrant upregulation of ST-2 ligands.

In the IL-33/ST-2 intracellular signaling pathway, IL-33 binds the heterodimeric receptor complex that includes ST-2 and the IL-1R accessory protein (i.e., IL-1RAP). IL-33 is believed to induce signaling through the TIR domain of IL-1RAP. As a result of IL-33 binding, myeloid differentiation primary-response protein 88 (MYD88), IL-1R-associated kinase-1 (IRAK-1), and IL-1R-associated kinase-4 (IRAK-4) are recruited to the receptor complex and induce the activation of numerous signaling proteins, including nuclear factor-κB (NF-κB), inhibitor of NF-κB-α (IκBα), extracellular signal-regulated kinase-1 (ERK-1, also known as MAPK-3), extracellular signal-regulated kinase-2 (ERK-2, also known as MAPK-1), p38 (also known as MAPK-13) and JUN N-terminal kinase-1 (JNK1, also known as MAPK-8). In mast cells, the pathway further involves phospholipase D and sphingosine kinase, which can lead to the production of interleukin-1-beta (IL-1), interleukin-3 (IL-3), interleukin-6 (IL-6), tumor necrosis factor (TNF), CXC-chemokine ligand 2 (CXCL2), CC-chemokine ligand 2 (CCL2), CC-chemokine ligand 3 (CCL3), prostaglandin D2 (PGD2) and leukotriene B4 (LTB4). However, IL-33 also induces the production of interleukin-5 (IL-5), interleukin-13 (IL-13), CC-chemokine ligand 5 (CCLS), CC-chemokine ligand 17 (CCL17) and CC-chemokine ligand 24 (CCL24) by mast cells and T cells, which is mediated by an NF-κB-independent mitogen-activated protein kinase (MAPK)-dependent pathway (see Liew et al., supra). By increasing the expression of sST-2, serelaxin interferes with the above signaling cascade of IL-33. In one embodiment, this reduces or modulates the production of IL-1β, IL-3, IL-6, TNF, CXCL2, CCL2, CCL3, prostaglandin D2 and leukotriene B4 by mast cells as well as the production of IL-5, IL-13, CCLS, CCL17 and CCL24 by mast cells and T cells. As a result, inflammation in the tissue is reduced. In another embodiment, this substantially suppresses or inhibits most of the production of IL-1β, IL-3, IL-6, TNF, CXCL2, CCL2, CCL3, PGD2 and LTB4 by mast cells as well as the production of IL-5, IL-13, CCLS, CCL17 and CCL24 by mast cells and T cells. As a result, inflammation in the tissue is reduced or substantially down-regulated.

The ST-2 gene has been cloned in mice, rats, humans, and chickens. The molecular mechanism responsible for the transcriptional regulation of the ST-2 gene in TH2 cells includes proximal and distal promoters. With respect to promoter usage, the distal promoter dominates over the proximal promoter. GATA consensus sites have been found in both the mouse and human ST-2 genes of the distal promoter region. A region of approximately 100 base pair (bp) upstream of the transcription start site, containing two GATA consensus sites, is critical for the expression of the sST-2 gene. The GATA-3 transcription factor (i.e., an important factor for T-cell lineage development as well as the transcriptional regulation of TH2 cell-specific cytokine genes such as IL-4, IL-5, and IL-13) binds to a single GATA site in the critical region of the sST-2 distal promoter, which activates the expression of the sST-2 gene in TH2-type cells that have been stimulated with cAMP. Moreover, the expression of sST-2 mRNA is temporarily increased at three hours after stimulation with cAMP (Hayakawa et al. (2005) Biochimica et Biophysica Acta. 1728:53-64). This correlates with serelaxin being a strong inducer of cAMP. Serelaxin binds with high affinity to its serelaxin family peptide receptor 1 (RXFP1), which leads to induction of cAMP (Du et al. (2009) Nature Reviews 198:1-11). Without wanting to be bound by theory, it is contemplated that serelaxin may induce sST-2 expression via cAMP.

Treatment of Inflammation with Serelaxin

Membrane-bound ST-2 is selectively expressed in TH2 cells and mast cells, wherein ligand binding promotes TH2 cell activity. Ligands (e.g., IL-33) are known to have a dual role in diseases, protecting against atherosclerosis and helminth infection but also exacerbating TH2 and mast cell mediated inflammatory diseases. The soluble ST-2 (sST-2) receptor has been identified as a potential therapeutic target for treating inflammatory diseases. However, prior to the present disclosure, such therapy was considered untenable because of the risk that inhibiting the ST-2 intracellular signaling pathway would increase cardiac morbidity by eliminating the cardioprotective effect (Kakkar and Lee (2008) Nature Reviews 7:827-840). Notably, the inventor has found that serelaxin targets the ST-2 intracellular signaling pathway and provides therapy for inflammatory diseases without the associated cardiac risk of previously recognized anti-inflammatory therapeutics. Serelaxin acts as a vasomodulator and has favorable hemodynamic effects, greatly reducing the risk of adverse cardiac effects (Teerlink et al. (2009) Lancet 373:1429). In one embodiment, serelaxin reduces and/or modulates proinflammatory cytokines via the ST-2/IL-33 intracellular signaling pathway. In another embodiment, serelaxin substantially suppresses or inhibits proinflammatory cytokines via the ST-2/IL-33 intracellular signaling pathway.

The ST-2 intracellular signaling pathway is present in the lung (smooth muscle and epithelium lining the bronchi and small airways), peripheral blood leukocytes (TH2 lymphocytes and macrophages), skin, stomach, brain, spinal cord, joints, heart, and blood vessel endothelium. IL-33 induces TH2 cytokines in T cells and modulates a wide range of physiological responses, including but not limited to, the antigen/allergen response, autoimmunity, organ fibrosis and cardiac injury. IL-33 binds to and activates ST-2 signaling in many cell types involved in the immune response and plays a pathogenic role in a broad spectrum of immune-related diseases. In fact, IL-33 is involved in pathological states as varied as asthma, sepsis, rheumatoid arthritis, skin disorders, atherosclerosis, collagen vascular diseases and heart failure. The ST-2 signaling pathway is a potent inducer of proinflammatory cytokines in mast cells, basophils and eosinophils, which are all cellular mediators of allergy, asthma and septic shock. Ligand binding to ST-2 can also amplify activation of macrophages and dendritic cells.

The applicants have investigated the effect of serelaxin on the IL-33/ST-2 intracellular signaling pathway and have unexpectedly found that serelaxin up-regulates the soluble ST-2 (sST-2) receptor, which is also known as the decoy receptor for IL-33. sST-2 has been shown to have an anti-inflammatory effect in diseases driven by a TH2 immune response and/or mediated by IL33. These diseases include, but are not limited to, pleural malignancy, sepsis, trauma, wound healing, atopic allergy, anaphylaxis, autoimmume encephalomyelitis, eosinophilic airway hyperresponsiveness, CNS hypoxia/vascular damage, hypernociception, rheumatoid arthritis, multiple sclerosis (MS), ankylosing spondylitis (AS), inflammatory bowel disease, asthma, gout, myositis, Sjögren's syndrome, systemic lupus erythematosus (SLE), vasculitis, eczema, dermatitis, scleroderma, poison ivy, acne, hives, and psoriasis. Because serelaxin leads to a reduction and/or modulation and/or inhibition in inflammation via the IL-33/ST-2 intracellular signaling system, it can be used in the treatment of these diseases. Subjects for whom therapy with serelaxin is efficacious can be identified by testing them for a low level of sST-2, an elevated level of IL-33, or a combination of the two. In one embodiment, subjects for whom therapy with serelaxin is efficacious are identified by testing such subjects for low circulating levels of sST-2. In another embodiment, subjects for whom therapy with serelaxin is efficacious are identified by testing them for elevated circulating levels of IL-33. In yet another embodiment, subjects for whom therapy with serelaxin is efficacious are identified by testing them for low circulating levels of sST-2 as well as elevated circulating levels of IL-33.

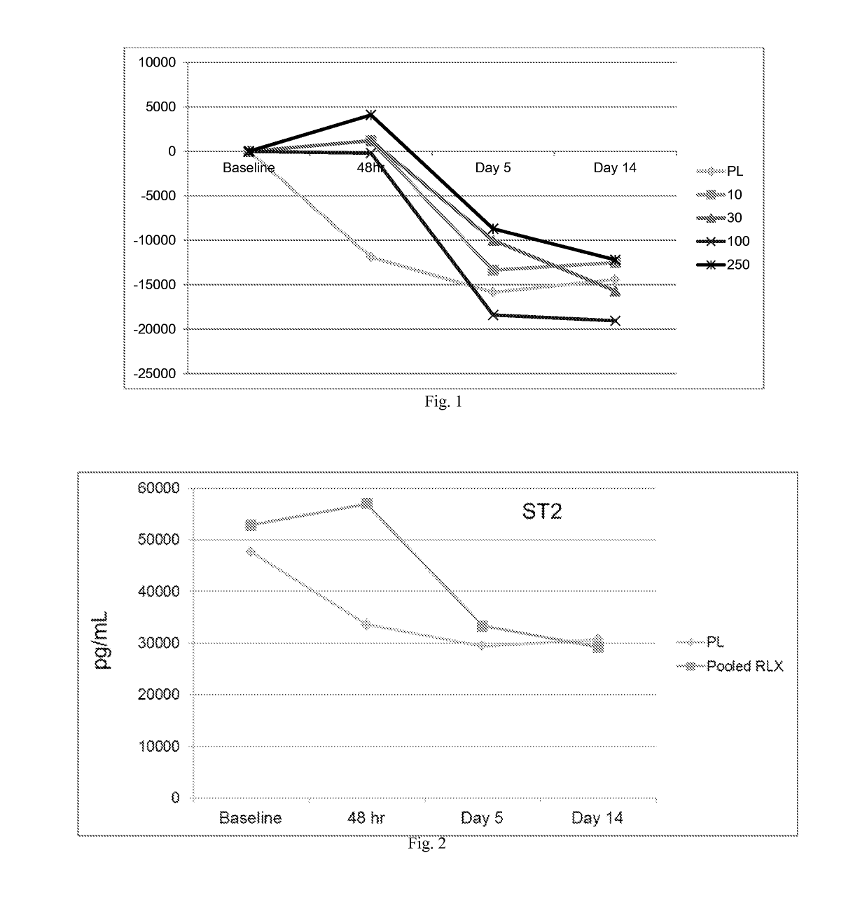

More specifically, the inventor has shown that serelaxin transiently upregulates sST-2 (see FIGS. 1, 2 and 3). As can be seen in FIG. 1, at 48 hours of serelaxin treatment, all treated groups were significantly different from the placebo group (i.e., the placebo group decreased 30% from baseline). At baseline, geometric means across treatment groups ranged from 47 ng/ml to 60 ng/ml, i.e., 55 percent of the treated subjects had sST-2 concentrations that exceeded the normal range of 33.5 ng/ml in females and 49.3 ng/ml in males. At 48 hours, the placebo group showed a 30% decrease in sST-2 concentrations while all serelaxin groups showed an increase from baseline (p<0.05 for all serelaxin groups vs. placebo). At days 5 and 14, the placebo and all serelaxin groups showed significant decreases from baseline in sST-2 by 35 to 50 percent, which was consistent with a decrease in inflammatory state in all groups. Thus, serelaxin transiently upregulates sST-2, which in turn leads to a reduction and/or modulation of inflammation. In one embodiment, transiently upregulated sST-2 binds to and inhibits IL-33. In another embodiment the transiently upregulated sST-2 reduces and/or modulates IL-33-mediated inflammation. In another embodiment, sST-2 binds to and inhibits or down-regulates IL-33, wherein IL-33 is naturally present in high levels in an inflammatory environment. Because IL-33 is such a potent inducer of proinflammatory cytokines and chemokines by mast cells, particularly IL-1, IL-6, IL-13, TNF, CCL2 and CCL3, its inhibition or down-regulation prevents induction of degranulation of IgE-primed mast cells and reduces mast cell maturation and survival. IL-33 also normally activates basophils that stimulate additional cytokines and chemokines and potently induces eosinophils and up-regulates the expression of adhesion molecules. Thus, IL-33 inhibition or down-regulation prevents or significantly reduces the induction of eosinophils and further down-regulates the expression of adhesion molecules. Since mast cells, eosinophils and adhesion molecules play important roles in allergic reaction, serelaxin can be therapeutically employed to treat allergy, asthma, septic shock and other disorders via the inhibition of IL-33.

For example, the IL-33/ST-2 intracellular signaling system has been implicated in a wide array of autoimmune disorders, including rheumatoid arthritis and joint disease. Human subjects express high levels of IL-33 in rheumatic synovial fluid and sST-2 levels are elevated in the sera of subjects with systemic lupus erythematosis, progressive systemic sclerosis, and Wegener's granulomatosis, suggesting an important role for sST-2 mediated inhibition of IL-33 in a spectrum of autoimmune diseases (Kakkar and Lee, supra). It is noteworthy that blocking the function of IL-33 in a mouse model (i.e., either by sST-2 administration or sST-2 gene deletion or administration of a specific antibody to ST-2) results in a decreased disease severity in a collagen-induced arthritis mouse model (Liew et al., supra). In addition, others have shown similar results with an sST2-Fc fusion protein in another murine model of collagen-induced arthritis, wherein a short term administration of the sST2-Fc fusion protein significantly reduced disease severity compared with controls (Leung et al. (2004) The Journal of Immunology 173:145-150). Serelaxin can be used to induce sST-2 such that IL-33 can be reduced or inhibited in autoimmune disorders like arthritis that are characterized by T-cell dominant inflammation. In one embodiment, serelaxin is used to induce sST-2 such that ST-2 ligands such as IL-33 are reduced in autoimmune disorders. In another embodiment, serelaxin is used to induce sST-2 such that ST-2 ligands such as IL-33 are substantially suppressed or inhibited in autoimmune disorders.

In the lung, ST-2 signaling is involved in the immune response. Exposure to ligand results in epithelial hypertrophy and mucus accumulation, indicators of an inflammatory process in bronchi. For example, IL-33 is expressed at higher levels in asthmatic subjects. In keeping with the decoy function of sST-2, gene transfer of sST-2 to mice markedly attenuates airway inflammation in response to an immune challenge and pre-exposure to sST-2 lowers production of TH2 cytokines in a mouse model of allergen-induced pulmonary inflammation. In human subjects, serum levels of sST-2 are elevated in correlation with acute exacerbations of asthma and in subjects with acute eosinophilic pneumonia. Interestingly, sST-2 gene and protein expression can be induced in an alveolar macrophage (MA) cell line in response to proinflammatory stimuli (e.g., LPS, IL-Iβ, Il-6, TNF-α) as well as in an LPS-induced mouse lung injury model, while ST-2 gene expression appears to be constitutive and does not change before or after stimulation. In addition, pretreatment with sST-2 protein results in the down-regulation in gene and protein expression of proinflammatory cytokines including IL-1α, IL-6, and TNF-α in LPS-stimulated MA cells. This indicates that sST-2 can suppress production of proinflammatory cytokines that would otherwise lead to acute lung injury (Oshikawa et al. (2002) Biochemical and Biophysical Research Communications 299:18-24). Since serelaxin up-regulates sST-2 it provides a new therapeutic treatment for inflammation of the lung (e.g., asthma). Although, serelaxin has been implicated as potentially reducing lung fibrosis it was not believed to play any role in lung inflammation (Royce et al. (2009) Endocrinology 150: 2692-2699). Thus, the finding that serelaxin may reduce or suppress the production of proinflammatory cytokines via sST-2 in the lung is surprising and unexpected. Thus, in one embodiment, serelaxin is used to induce sST-2 such that ST-2 ligands are reduced in pulmonary inflammation. In another embodiment, serelaxin is used to induce sST-2 such that ST-2 ligands are substantially suppressed or inhibited in pulmonary inflammation.

IL-33 is primarily expressed by cells of barrier tissues and plays an important part in specific disorders of the skin. For example, in atopic dermatitis (AD), TH2 cytokines characterize the inflammatory response. Furthermore, there is increased expression of IL-33 and sST-2 in AD skin after allergen or staphylococcal enterotoxin B (SEB) exposure in mice. In addition, skin fibroblasts, cultured keratinocytes, primary macrophages, and HUVEC endothelial cells produce IL-33 in response to a combined stimulation of tumor necrosis factor-α and IFN-γ. The increased expression of IL-33 and sST-2 that is caused by irritants, allergen, or SEB challenges can be suppressed by topical tacrolimus treatment. There is upregulation of IL-33 and sST2 in the lesional AD skin by certain triggering factors such as allergen exposure, irritants, scratching, and bacterial and viral infections (Savinko et al. (Jan. 26, 2012) Journal of Investigative Dermatology; on-line; IL-33 and ST-2 in Atopic Dermatitis: Expression Profiles and Modulation by Triggering Factors). This shows that the IL-33-sST2 interaction plays a significant role in the pathogenesis and disease severity of AD. This in turn supports the inventor's findings that the increased expression of ST-2 ligands can be modulated and substantially reduced with serelaxin through the up-regulation of sST-2. Hence, in one embodiment, serelaxin is used to induce sST-2 such that ST-2 ligands are reduced in inflammatory skin disorders. In another embodiment, serelaxin is used to induce sST-2 such that ST-2 ligands are substantially suppressed or inhibited in inflammatory skin disorders.

In sepsis and trauma, subjects have elevated serum levels of IL-4 and IL-10, and decreased levels of TH1 cytokines, which is often a sign of a potentially unfavorable disease prognosis. Subjects that are admitted to an emergency room or intensive care unit with a diagnosis of sepsis or after experiencing significant trauma also show high serum levels of sST-2, which is likely a further sign of pathogenesis. In a mouse sepsis model, the administration of sST-2 results in reduced serum levels of IL-6, IL-12 and TNFα as well as increased survival (Kakkar and Lee, supra). This suggests that sST-2 can modulate and reduce proinflammatory stimuli in sepsis and trauma. Hence, treatment with serelaxin can provide a new therapeutic avenue to address these disease processes. Moreover, sST-2 is likely to correlate with the prognosis and/or survival of subjects suffering from sepsis and/or trauma. Thus, subjects who are admitted to the hospital with sepsis or severe trauma that also have elevated levels of sST-2 are likely good candidates for serelaxin treatment. Hence, in one embodiment, serelaxin is used to induce sST-2 such that ST-2 ligands are reduced in diseases such as sepsis and trauma. In another embodiment, serelaxin is used to induce sST-2 such that ST-2 ligands are substantially suppressed or inhibited in diseases such as sepsis and trauma.

The ST-2 signaling is also involved in the fibroproliferative response to tissue injury and fibroproliferative diseases. For example, subjects with an acute exacerbation of pulmonary fibrosis exhibit elevated serum levels of sST-2. When mice are exposed to a hepatotoxin (i.e., hepatotoxin carbon tetrachloride) they exhibit an accelerated post-injury fibrotic response when treated with an sST-2-Fc fusion protein, wherein the effect seems to be mediated by the ability of the fusion protein to block TLR-4 mediated signaling. This is consistent with the involvement of sST-2 in TLR-4 signaling in a model of LPS-induced sepsis (Kakkar and Lee, supra). Since the fibrotic response to injury is a feature of most tissues, serelaxin therapy finds wide applicability in fibroliferative diseases. In one embodiment, serelaxin is used to induce sST-2 such that ST-2 ligands are reduced in fibroliferative diseases. In another embodiment, serelaxin is used to induce sST-2 such that ST-2 ligands are substantially suppressed or inhibited in fibroliferative diseases.

Atopic allergy and anaphylaxis result in higher levels of IgE antibodies. A receptor through which IgE antibodies activate proinflammatory cytokines is the high affinity Fcε receptor I (FcεRI). IL-33 activates mast cells and induces degranulation after IgE sensitization has occurred. However, the expression of IL-33 alone cannot usually trigger anaphylactic shock or an acute allergic response. Rather, IL-33 functions in an additive manner to further worsen these conditions. Still, the ST-2 signaling system is a potential therapeutic target (Liew et al., supra). In one embodiment, serelaxin is used to induce sST-2 such that ST-2 ligands (e.g., IL-33) are reduced in diseases related to allergy and/or anaphylaxis. In another embodiment, serelaxin is used to induce sST-2 such that ST-2 ligands are substantially suppressed or inhibited in diseases related to allergy and/or anaphylaxis. For example, when IL-33 is reduced then the condition of anaphylactic shock is ameliorated because IL-33 can no longer worsen the condition. Similarly, the same holds true for an acute allergic response, which can be stabilized through the use of serelaxin.

The IL-33/ST-2 system is also involved in the central nervous system (CNS) and nociception (transmission of pain). For example, IL-33 is produced in the CNS where it activates microglia and is believed to function as a pro-inflammatory mediator in the pathophysiology of the CNS. The IL-33 receptor is expressed mainly in microglia and astrocytes. However, the IL-33 ligand is produced by endothelial cells and astrocytes but not by microglia or neurons. In the CNS, IL-33 induces the proliferation of microglia and pro-inflammatory cytokines, including IL-1β, TNFα, and IL-10. IL-33 also induces chemokines and nitric oxide production (Yasuoka et al. (2011) Brain Research 1385:8-17). Treating mice with IL-33 exacerbates experimental autoimmune encephalomyelitis (EAE). Viral infection in the mouse CNS induces IL-33 mRNA expression, suggesting that IL-33 plays a role in the host defense of the CNS. There is also evidence that IL-33 is involved in CNS hypoxia and vascular damage because subjects with subarachnoid hemorrhage show an increased expression of ST-2 on the cells in the cerebrospinal fluid. Also, IL-33 is implicated in the peripheral nervous system where it can induce inflammatory pain. Hypernociception is the sensitization of nociceptors (i.e., pain receptors) that are responsible for transmitting pain. For example, cutaneous and articular hypernociception can be induced in mice by local administration of IL-33. In an antigen-induced cutaneous hypernociception model, pain can be attenuated by treatment with sST-2 (Liew et al., supra). Thus, treatment with serelaxin can be used to decrease pain transmission throughout the body. In one embodiment, serelaxin is used to induce sST-2 such that ST-2 ligands (e.g., IL-33) are reduced in hypernociception and/or other diseases related to the CNS. In another embodiment, serelaxin is used to induce sST-2 such that ST-2 ligands (e.g., IL-33) are substantially suppressed or inhibited in hypernociception and/or other diseases related to the CNS.

IL-33 is known to be up-regulated in chronically inflamed tissues such as the intestines of subjects suffering from Crohn's disease (CD) and the synovium of rheumatoid arthritis subjects. IL-33 has been associated with pathologic changes in the GI tract, including esophagus, small and large intestine, and spleen (Schmitz et al. (2005) Immunity 23:479-490). It is widely accepted that inflammatory bowel syndrome (IBD), including CD and ulcerative colitis (UC) are inflammatory disorders that are largely the result of an imbalance of inflammatory agents. For example, imbalances in IL-1 and IL-18 have been implicated in the pathogenesis of UC and CD and sST-2 has been suggested to have some anti-inflammatory properties because it functions as a decoy receptor for IL-33. It has also been established that blocking proinflammatory cytokines via TNF therapy is an effective way to down-regulate the mucosal inflammation of IBD. ST-2 signaling is thought to be activated in IBD and there is a strong rationale for anti-ST-2 ligand strategies to treat IBD (Pastorelli et al. (2010) PNAS 107:8017-8022). Thus, serelaxin can be used to treat inflammatory diseases of the GI tract. In one embodiment, serelaxin is used to induce sST-2 such that its ligands are reduced in inflammation of the GI tract. In another embodiment, serelaxin is used to induce sST-2 such that its ligand (e.g., IL-33) is substantially suppressed or inhibited in inflammation of the GI tract.

The ST-2 gene is upregulated in cardiovascular disease, it is induced by mechanical stimulation of cardiomyocytes and is a known biomarker for mechanical overload of the heart. Activation of the transmembrane form ST-2 by IL-33 has a cardioprotective effect, conversely, sST-2 is elevated in myocardial infarction and heart failure subjects (Kakkar and Lee, supra). As such sST-2 correlates with mortality and is sometimes referred to as a prognostic biomarker of mortality, independent of BNP, in subjects presenting with acute dyspnea (Januzzi et al. (2007) J. Am. Coll. Cardiol. 50:607-613). However, IL-33 is also angiogenic and causes vascular permeability via nitric oxide (NO). IL-33 is capable of directly activating endothelial cells (ECs), which results in promoting angiogenesis and hyperpermeability. Thus, IL-33 contributes to the pathogenesis of angiogenesis-dependent inflammatory vascular disease (Choi et al. (2009) Blood 114:3117-3126) in spite of its sometimes desirable cardio-protective effect. While a small amount of IL-33 may be cardio-protective to some subjects, too much IL-33 (as in vascular inflammation) is destructive to the vasculature. Thus, an agent that could reduce access IL-33 would be beneficial to subjects that may otherwise suffer from increasing vascular inflammation, overriding any cardio-protective effect of IL-33. Serelaxin has been shown to be beneficial to AHF subjects because of it vasomodulatory role. Herein, serelaxin acts as a vasomodulator with favorable hemodynamic effects, greatly reducing the risk of adverse cardiac events (Teerlink et al., supra). Serelaxin may further improve the outcome of heart subjects by reducing ST-2 ligand mediated vascular inflammation. This is unexpected because the ST-2 ligand IL-33 has always been believed to be mostly cardio-protective in heart subjects. However, serelaxin induces sST-2, thereby modulating and reducing the IL-33 mediated angiogenesis and vascular permeability. The positive effects of angiogenesis are not lost, however, because serelaxin continues to induce angiogenesis by inducing VEGF. Serelaxin may be the first treatment that can effectively reduce vascular inflammation via sST-2 without compromising the cardio-protective effect. This is likely due to the fact that too much IL-33 is destructive to the vasculature and serelaxin can be used to modulate it down to a lower and more desirable level, and, thus, a protective level. In an embodiment, serelaxin is used to induce sST-2 such that IL-33 is reduced in vascular inflammation.

In summary, serelaxin is capable of affecting the ST-2 signaling pathway by transiently up-regulating sST-2. Therefore, serelaxin is effective in treating inflammatory disorders because it induces sST-2 expression and thereby promotes the reduction or down-regulation of ST-2 ligands in specific tissues or organs, resulting in a reduction of proinflammatory cytokines and inflammation. As such, the applicants have devised a method of treating ST-2 ligand-mediated inflammation by administering serelaxin. More specifically, subjects are treated with a daily dose of pharmaceutically active serelaxin in an amount in a range of about 1 to 1000 μg/kg/day of subject body weight per day. Depending on the inflammatory condition, the dose may also be administered weekly or monthly. In one embodiment, the dosages of serelaxin are 10, 30, 100 or 250 μg/kg/day. These dosages result in serum concentrations of serelaxin of about 1, 3, 10, 30, 75 or 100 ng/ml. In one embodiment, pharmaceutically effective serelaxin or an agonist thereof is administered at about 30 μg/kg/day. In another embodiment, pharmaceutically effective serelaxin or an agonist thereof is administered at about 10 to about 250 μg/kg/day. In another embodiment, the administration of serelaxin is continued as to maintain a serum concentration of serelaxin of from about 0.5 to about 500 ng/ml, from about 0.5 to about 300 ng/ml, and from about 1 to about 50 ng/ml. In one embodiment, the administration of serelaxin is continued as to maintain a serum concentration of serelaxin of approximately 10 ng/ml. These serelaxin concentrations are predicted to ameliorate or reduce inflammation associated with inflammatory disorders, including, but not limited to pleural malignancy, sepsis, trauma, wound healing, atopic allergy, anaphylaxis, autoimmume encephalomyelitis, eosinophilic airway hyperresponsiveness, CNS hypoxia/vascular damage, hypernociception, rheumatoid arthritis, multiple sclerosis (MS), ankylosing spondylitis (AS), inflammatory bowel disease, asthma, gout, myositis, Sjögren's syndrome, systemic lupus erythematosus (SLE), vasculitis, eczema, dermatitis, scleroderma, poison ivy, acne, hives, and psoriasis.

Serelaxin Compositions and Formulations

Serelaxin, serelaxin agonists and/or serelaxin analogs are formulated as pharmaceuticals to be used in the methods of the disclosure. Any composition or compound that can stimulate a biological response associated with the binding of biologically or pharmaceutically active serelaxin (e.g., synthetic serelaxin, recombinant serelaxin) or a serelaxin agonist (e.g., serelaxin analog or serelaxin-like modulator) in order to transiently up-regulate sST-2 can be used as a pharmaceutical in the disclosure. General details on techniques for formulation and administration are well described in the scientific literature (see Remington's Pharmaceutical Sciences, Maack Publishing Co, Easton Pa.). Pharmaceutical formulations containing pharmaceutically active serelaxin can be prepared according to any method known in the art for the manufacture of pharmaceuticals. The formulations containing pharmaceutically active serelaxin or serelaxin agonists used in the methods of the disclosure can be formulated for administration in any conventionally acceptable way including, but not limited to, intravenously, subcutaneously, intramuscularly, sublingually, intranasally, intracerebrally, intracerebroventricularly, topically, orally, intravitrealy and via inhalation. Illustrative examples are set forth below. In one embodiment, serelaxin is administered intravenously or subcutaneously.

When serelaxin is delivered by intravenous or subcutaneous injection (e.g., infusion, bolus, pump), the formulations containing pharmaceutically active serelaxin or a pharmaceutically effective serelaxin agonist can be in the form of a sterile injectable preparation, such as a sterile injectable aqueous or oleaginous suspension. This suspension can be formulated according to the known art using those suitable dispersing or wetting agents and suspending agents, which have been mentioned above. The sterile injectable preparation can also be a sterile injectable solution or suspension in a nontoxic parenterally-acceptable diluent or solvent. Among the acceptable vehicles and solvents that can be employed are water and Ringer's solution, an isotonic sodium chloride. In addition, sterile fixed oils can conventionally be employed as a solvent or suspending medium. For this purpose any bland fixed oil can be employed including synthetic mono- or diglycerides. In addition, fatty acids such as oleic acid can likewise be used in the preparation of injectables.

Aqueous suspensions of the disclosure contain serelaxin in admixture with excipients suitable for the manufacture of aqueous suspensions. Such excipients include a suspending agent, such as sodium carboxymethylcellulose, methylcellulose, hydroxypropylmethylcellulose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethylene oxycetanol), a condensation product of ethylene oxide with a partial ester derived from a fatty acid and a hexitol (e.g., polyoxyethylene sorbitol mono-oleate), or a condensation product of ethylene oxide with a partial ester derived from fatty acid and a hexitol anhydride (e.g., polyoxyethylene sorbitan monooleate). The aqueous suspension can also contain one or more preservatives such as ethyl or n-propyl p-hydroxybenzoate, one or more coloring agents, one or more flavoring agents and one or more sweetening agents, such as sucrose, aspartame or saccharin. Formulations can be adjusted for osmolarity.

Oil suspensions can be formulated by suspending serelaxin in a vegetable oil, such as arachis oil, olive oil, sesame oil or coconut oil, or in a mineral oil such as liquid paraffin. The oil suspensions can contain a thickening agent, such as beeswax, hard paraffin or cetyl alcohol. Sweetening agents can be added to provide a palatable oral preparation. These formulations can be preserved by the addition of an antioxidant such as ascorbic acid.

Dispersible powders and granules of the disclosure suitable for preparation of an aqueous suspension by the addition of water can be formulated from serelaxin in admixture with a dispersing, suspending and/or wetting agent, and one or more preservatives. Suitable dispersing or wetting agents and suspending agents are exemplified by those disclosed above. Additional excipients, for example sweetening, flavoring and coloring agents, can also be present.

The pharmaceutical formulations of the disclosure can also be in the form of oil-in-water emulsions. The oily phase can be a vegetable oil, such as olive oil or arachis oil, a mineral oil, such as liquid paraffin, or a mixture of these. Suitable emulsifying agents include naturally-occurring gums, such as gum acacia and gum tragacanth, naturally occurring phosphatides, such as soybean lecithin, esters or partial esters derived from fatty acids and hexitol anhydrides, such as sorbitan mono-oleate, and condensation products of these partial esters with ethylene oxide, such as polyoxyethylene sorbitan mono-oleate.

Administration and Dosing Regimen of Serelaxin Formulations

The formulations containing pharmaceutically active serelaxin or pharmaceutically effective serelaxin agonist used in the methods of the disclosure can be administered in any conventionally acceptable way including, but not limited to, intravenously, subcutaneously, intramuscularly, sublingually, intranasally, intracerebrally, intracerebroventricularly, topically, orally, intravitrealy and via inhalation. Administration will vary with the pharmacokinetics and other properties of the drugs and the subjects' condition of health. General guidelines are presented below.

The methods of the disclosure reduce inflammation associated with IL-33-mediated inflammatory disorders or other conditions. The amount of serelaxin alone or in combination with another agent or drug that is adequate to accomplish this is considered the therapeutically effective dose. The dosage schedule and amounts effective for this use, i.e., the “dosing regimen,” will depend upon a variety of factors, including the stage of the inflammatory disorder or condition, the severity of the inflammatory disorder or condition, the severity of the adverse side effects, the general state of the subject's health, the subject's physical status, age and the like. In calculating the dosage regimen for a subject, the mode of administration is also taken into consideration. The dosage regimen must also take into consideration the pharmacokinetics, i.e., the rate of absorption, bioavailability, metabolism, clearance, and the like. Based on those principles, serelaxin can be used to treat inflammation in individuals afflicted with ST-2 ligand-mediated (e.g., IL-33-mediated) inflammatory disorders.

The disclosure also provides the use of serelaxin in the manufacture of a medicament for treating ST-2 ligand-mediated inflammation, wherein the medicament is specifically prepared for treating afflicted individuals. Further contemplated is the use of serelaxin in the manufacture of a medicament for treating ST-2 ligand-mediated inflammation, wherein the subject has previously (e.g., a few hours before, one or more days before, etc.) been treated with a different drug. In one embodiment, the other drug is still active in vivo in the subject. In another embodiment, the other drug is no longer active in vivo in the subject.

The state of the art allows the clinician to determine the dosage regimen of serelaxin for each individual subject. As an illustrative example, the guidelines provided below for serelaxin can be used as guidance to determine the dosage regimen, i.e., dose schedule and dosage levels, of formulations containing pharmaceutically active serelaxin administered when practicing the methods of the disclosure. As a general guideline, it is expected that the daily dose of pharmaceutically active serelaxin (e.g., synthetic, recombinant, analog, agonist, etc.) is typically in an amount in a range of about 1 to 1000 μg/kg of subject body weight per day. In one embodiment, the dosages of serelaxin are 10, 30, 100 or 250 μg/kg/day. In another embodiment, these dosages result in serum concentrations of serelaxin of about 1, 3, 10, 30, 75 or 100 ng/ml. In one embodiment, pharmaceutically effective serelaxin or an agonist thereof is administered at about 30 μg/kg/day. In another embodiment, pharmaceutically effective serelaxin or an agonist thereof is administered at about 10 to about 250 μg/kg/day. In another embodiment, the administration of serelaxin is continued as to maintain a serum concentration of serelaxin of from about 0.5 to about 500 ng/ml, more preferably from about 0.5 to about 300 ng/ml, and most preferably from about 1 to about 10 ng/ml. In one embodiment, the administration of serelaxin is continued as to maintain a serum concentration of serelaxin of about 10 ng/ml or greater. Thus, the methods of the present disclosure include administrations that result in these serum concentrations of serelaxin. These serelaxin concentrations can ameliorate or reduce inflammation associated with inflammatory disorders, including, but not limited pleural malignancy, sepsis, trauma, wound healing, atopic allergy, anaphylaxis, autoimmume encephalomyelitis, eosinophilic airway hyperresponsiveness, CNS hypoxia/vascular damage, hypernociception, rheumatoid arthritis, multiple sclerosis (MS), ankylosing spondylitis (AS), inflammatory bowel disease, asthma, gout, myositis, Sjögren's syndrome, systemic lupus erythematosus (SLE), vasculitis, eczema, dermatitis, scleroderma, poison ivy, acne, hives, and psoriasis. Furthermore, these serelaxin concentrations can ameliorate or reduce inflammation in disorders that are not traditionally known as inflammatory disorders such as cancer. Depending on the subject, the serelaxin administration is maintained for as specific period of time or for as long as needed to achieve stability in the subject. For example, the duration of serelaxin treatment is preferably kept at a range of about 4 hours to about 96 hours, more preferably 8 hours to about 72 hours, depending on the subject, and one or more optional repeat treatments as needed.

Single or multiple administrations of serelaxin formulations may be administered depending on the dosage and frequency as required and tolerated by the subject who suffers from ST-2 ligand-mediated inflammation. The formulations should provide a sufficient quantity of serelaxin to effectively ameliorate the condition. A typical pharmaceutical formulation for intravenous subcutaneous administration of serelaxin would depend on the specific therapy. For example, serelaxin may be administered to a subject through monotherapy (i.e., with no other concomitant medications) or in combination therapy with another medication. In one embodiment, serelaxin is administered to a subject daily as monotherapy. In another embodiment, serelaxin is administered to a subject daily as combination therapy with another drug. Notably, the dosages and frequencies of serelaxin administered to a subject may vary depending on age, degree of illness, drug tolerance, and concomitant medications and conditions.

In some embodiments, serelaxin is provided as a 1.0 mg/ml solution (3.5 ml in 5.0 ml glass vials). Placebo, which is identical to the diluent for serelaxin, is provided in identical vials. Serelaxin or placebo can be administered intravenously or subcutaneously to the subject in small volumes using a syringe pump in combination with normal saline in a piggyback configuration. Compatible tubing and a 3-way stopcock, which have been tested and qualified for use with serelaxin are used to administer the serelaxin formulation. Doses are administered on a weight basis and adjusted for each subject by adjusting the rate of serelaxin drug delivered by, for example, the infusion pump.

### MODES FOR CARRYING OUT THE INVENTION

The following specific examples are intended to illustrate the disclosure and should not be construed as limiting the scope of the claims.

### Example 1

### Sample Collection from Subjects with Heart Failure

Samples were collected from subjects enrolled in a multi-center, randomized, double-blind, placebo-controlled clinical trial, which was conducted to determine the safety and efficacy of serelaxin (recombinant human serelaxin) in subjects with heart failure, as described in Teerlink et al. (supra).

### Example 2

### Clinical Sample Analysis

Serum was collected from 218 subjects (supra) for whom a baseline level of sST-2 as well as a post-serelaxin treatment level of sST-2 was determined sST-2 was found at a lower limit of quantitation of 3.1 ng/ml and an upper limit of quantitation of 200 ng/ml. sST-2 was measured via the EIA kit (see Example 7 for more detail) and exact clinical measurements are summarized in Table 1 (see attached Table 1).

### Example 3

### Serelaxin Induced a Transient Increase in sST-2

FIG. 1 shows a graph that depicts the change from baseline in median sST-2 concentrations with time in subjects treated with serelaxin and placebo. Whereas sST-2 declined in the placebo group at 48 hours, the serelaxin groups showed a transient increase in sST-2 levels at 48 hours. The difference between the placebo group and each of the serelaxin dose groups was statistically significant at 48 hours. However, this difference did not persist, as the changes from baseline decreased in all groups by Day 5 and Day 14.

More specifically, at 48 hours of serelaxin treatment, all treated groups were significantly different from the placebo group (i.e., the placebo group decreased 30% from baseline). At baseline, geometric means across treatment groups ranged from 47 ng/ml to 60 ng/ml, i.e., 55 percent of the treated subjects had sST-2 concentrations that exceeded the normal range of 33.5 ng/ml in females and 49.3 ng/ml in males. At 48 hours, the placebo group showed a 30% decrease in sST-2 concentrations while all serelaxin groups showed an increase from baseline (p<0.05 for all serelaxin groups vs. placebo). At days 5 and 14, the placebo and all serelaxin groups showed significant decreases from baseline in sST-2 by 35 to 50 percent. This established that serelaxin transiently upregulates sST-2.

FIG. 2 illustrates the serelaxin groups as pooled compared to placebo, i.e., it shows the change from baseline in median sST-2 concentrations with time in subjects in the placebo group and the pooled serelaxin group.

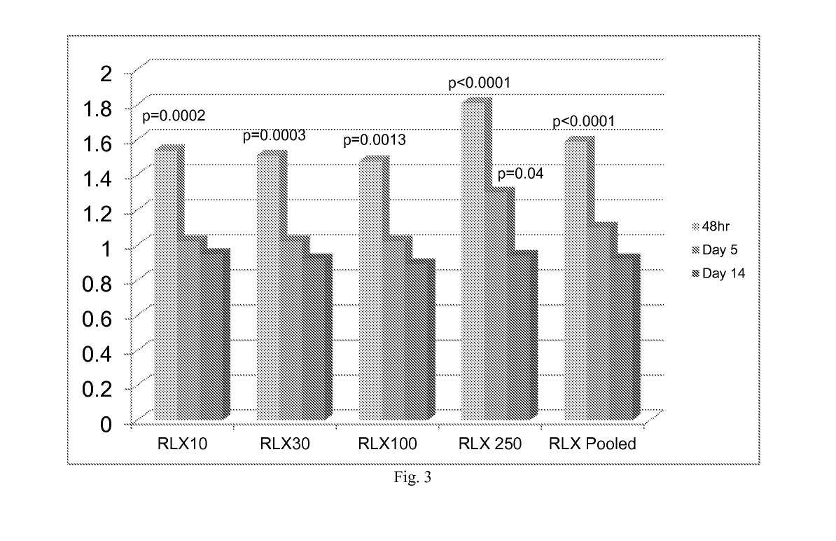

FIG. 3 depicts a between-treatment analysis of sST-2 by visit, comparing a change from baseline in sST-2 geometric means between the serelaxin groups compared to the placebo group at 3 time points. At 48 hours of treatment, change from baseline in all serelaxin groups, as well as the pooled serelaxin group, were significantly different from the change from baseline in the placebo group. The differences subsided so that by Day 5, changes from baseline in 3 of the 4 serelaxin groups, as well as the pooled serelaxin group, were no different than change from baseline in the placebo group. By Day 14, the changes from baseline in all of the serelaxin groups were not different from change in the placebo group. As above, this establishes that serelaxin transiently up-regulates sST-2.

### Example 4

### Treatment of Inflammation with Serelaxin

Subjects that experience inflammation with elevated levels of IL-33 and/or low levels of sST-2 can be treated with serelaxin in order to reduce their tissue inflammation. In one embodiment, serelaxin prevents or ameliorates consequences of inflammation. Subjects that are eligible for serelaxin treatment could be enrolled in a randomized study to receive in a double blind manner, either IV placebo or serelaxin at 10, 30, 100 or 250 mg/kg/day for 48 hours. Alternatively, the subjects can be treated with serelaxin in the same manner in addition to standard therapy for inflammation at the discretion of the physician. Other routes of administration or dosages may also be evaluated during a study. The placebo used for the study should be the same solution as the diluent used to prepare the 100 mg/kg/day dose of serelaxin. The subjects are assessed regularly during the serelaxin treatment for signs and symptoms in order to monitor their inflammatory state, for example, at 6 h, 12 h, 24 h, 48 h after initiation of the serelaxin therapy and at days 3, 4, 5 and 14 or as otherwise held necessary by the supervising physicians. When the study concludes, subjects are to be tested for the levels of ST-2 ligands (e.g., IL-33) and sST-2 via the EIA assay, see Example 7) to reevaluate the inflammatory state of the subjects. In addition, blood samples may be taken from the subjects to test for the presence of inflammatory agents as well as circulating levels of ST-2 ligands (e.g., IL-33) and/or sST-2 which can be quantified, normalized and compared to the original measurements. Subjects who benefit from serelaxin treatment will ultimately show a reduction in one or more inflammatory agents including, but not limited to, IL-1β, IL-3, IL-5, IL-6, IL-13, IL-33, TNF, CXCL2, CCL2, CCL3, CCLS, CCL17, CCL24, PGD2, and LTB4. Subjects who benefit from serelaxin treatment would, thus, experience a decrease in ST-2 ligand-mediated inflammation. Numerous nonclinical toxicology studies have shown that serelaxin is safe when administered over a wide range of doses and for up to six months of continuous treatment. Therefore, it can be reasoned that serelaxin does not interfere with normal homeostatic mechanisms involving ST2 expression in immune cells (Kakkar et al., supra), which may be of concern if sST-2 were to be administered directly.

### Example 5

### Identification of Subjects that Benefit from Serelaxin Treatment

The inventor proposes that serelaxin acts by up-regulating sST-2, which functions as a decoy receptor and reduces the amount of ST-2 ligands (e.g., IL-33) available in an inflammatory environment. For example, IL-33 is a potent activator of proinflammatory agents, thus such agents would be down-regulated, reducing inflammation. In an embodiment, subjects with inflammation (e.g., subjects suffering from asthma, arthritis, joint disease, etc.) will have their circulating levels of ST-2 ligands such as IL-33 measured. If ST-2 ligand-mediated inflammation is present then ligand levels are expected to be higher than baseline levels. Thus, subjects with high circulating levels of ST-2 ligands would be identified as candidates for serelaxin treatment. Such subjects would benefit from reducing ST-2 ligands via serelaxin to ameliorate inflammation. In one embodiment the ST-2 ligand is IL-33. Notably, in severely compromised subjects, sST-2 levels may also be significantly up-regulated because of the natural immune system function. For example, in subjects with heart failure sST-2 levels can be as high as 50 ng/ml to 130 ng/ml or even higher. In such subjects both ST-2 ligands such as IL-33 and sST-2 should be measured. Heart failure subjects would benefit from reducing ST-2 ligands such as IL-33, to a level where they are protective but no longer inflammatory.

Subjects with inflammation and normal or only slightly decreased ST-2 levels are also candidates for serelaxin treatment. Normal sST-2 levels suggest that the decoy receptor is not yet up-regulated, giving IL-33 more potency in enhancing proinflammatory cytokines. The decoy is likely attempting to reduce IL-33-mediated inflammation but lacks potency. Subjects with low levels of circulating sST-2 would also be good candidates for serelaxin treatment because either the decoy receptor is not up-regulated or is not functioning, allowing IL-33 to continuously induce inflammatory agents.

### Example 6

### Healthy Donor sST-2 Reference

In order to establish a baseline reference of sST-2 levels in normal healthy individuals a self-reported-healthy-cohort has previously been determined and is available for reference comparison via the EIA Test Kit (see EIA Test Kit/PRESAGE sST-2 Assay from Critical Diagnostics, NY). The cohort includes 490 healthy donors that were equally distributed between the genders. Ages range from 18 to 84 of age. The cohort shows no bias based on age for sST-2 values (Kruskal-Wallis test; males=0.501, females=0.056) but sST-2 values as a function of gender differ significantly (Kruskal-Wallis test p<0.0001). Out of 490 donors, half are male and half are female. The median sST-2 concentration was determined to be about 18.8 ng/ml for the entire group, wherein the median for male was 23.6 ng/ml and the median for female was 16.2 ng/ml. However, based on the total analysis, the range of sST-2 for normal healthy males was determined to be 8.5 to 49.3 ng/ml, while the range of sST-2 for normal healthy females was determined to be 7.1 to 33.5 ng/ml. Thus, it is possible to compare sST-2 levels in subjects to these healthy standards as was done in Table 1. Table 1 includes a summary statistics for sST-2 by treatment group and visit, which extends to a full analysis of the subject population by the present inventor (see attached Table 1).

### Example 7

### Assay for Measuring Serelaxin Effect on sST-2 Levels

sST-2 levels were measured with the EIA Test Kit & PRESAGE sST-2 Assay (from Critical Diagnostics, NY) according to the manufacturer's instructions. Alternatively, other assays could be used such as the QUANTIKINE ST2/IL-1 R4 Immunoassay (from R&D Systems, Inc., MN).

The EIA test kit is a quantitative sandwich monoclonal ELISA in a 96 well plate format for measurement of sST-2 in serum or plasma. Diluted serum from 218 subjects was loaded into the appropriate wells in the anti-ST-2 antibody coated plate and incubated for the prescribed time. Following a series of steps, where reagents were washed from the plate, and additional reagents were added and subsequently washed out, the analyte sST-2 was detected by addition of a colorimetric reagent. The resulting signal was measured spectroscopically at 450 nm. The assay was conducted according to the parameters described in the assay description of the EIA test kit with all prescribed reagents and materials.

Various modifications and variations of the present disclosure will be apparent to those skilled in the art without departing from the scope and spirit of the disclosure. Although the disclosure has been described in connection with specific embodiments, it should be understood that the claims should not be unduly limited to such specific embodiments. Indeed, various modifications of the described modes for carrying out the disclosure, which are understood by those skilled in the art are intended to be within the scope of the claims.

1. A method of treating inflammation, comprising administering to a subject a pharmaceutically active serelaxin in a dose effective to cause transient up-regulation of soluble ST-2 in a tissue of said subject affected by inflammation, wherein said transient up-regulation of soluble ST-2 reduces proinflammatory cytokines in said tissue.

2. The method of claim 1, wherein said proinflammatory cytokines are induced by IL-33.

3. The method of claim 1, wherein said tissue is selected from the group consisting of lung tissue, skin tissue, joint tissue, nerve tissue and vascular tissue.

4. The method of claim 1, wherein said dose of pharmaceutically active serelaxin ranges from about 10 μg/kg/day to about 500 μg/kg/day.

5. The method of claim 1, wherein said inflammation comprises an inflammatory disorder selected from the group consisting of eosinophilic airway hyperresponsiveness, asthma, rheumatoid arthritis, multiple sclerosis (MS), ankylosing spondylitis (AS), inflammatory bowel disease, gout, myositis, Sjogren's syndrome, systemic lupus erythematosus (SLE), vasculitis, pleural malignancy, sepsis, trauma, wound healing, atopic allergy, anaphylaxis, autoimmune encephalomyelitis, CNS hypoxia, CNS vascular damage, hypernociception, eczema, dermatitis, scleroderma, poison ivy, acne, hives and psoriasis.

6. The method of claim 1, wherein said transient up-regulation of soluble ST-2 lasts from about 1 day to about 5 days.

7. The method of claim 1, wherein said soluble ST-2 binds 11-33.

8. The method of claim 7, wherein said soluble ST-2 functions as a decoy receptor lacking a transmembrane and/or cytoplasmic domain.

9. The method of claim 8, wherein said decoy receptor inhibits binding of said IL-33 to its transmembrane receptor ST-2.

10. The method of claim 5, wherein said inflammatory disorder is an IL-33-mediated immune response or an IL-33-mediated autoimmune disorder.

11. A method of treating inflammation, comprising:
(i) administering to a subject a pharmaceutically active serelaxin in a dose effective to cause transient up-regulation of soluble ST-2 in a tissue of said subject affected by inflammation, and
(ii) determining if said transient up-regulation of soluble ST-2 reduced proinflammatory cytokines in said tissue of said subject.

12. The method of claim 11, wherein said proinflammatory cytokines are induced by IL-33.

13. The method of claim 12, wherein said proinflammatory cytokines are reduced by inhibiting IL-33 with soluble ST-2.

14. The method of claim 13, wherein said reduced proinflammatory cytokines are evaluated by using an assay that measures serum levels of said proinflammatory cytokines in the peripheral blood of said subject.

15. The method of claim 11, wherein said tissue is selected from the group consisting of lung tissue, skin tissue, joint tissue, nerve tissue and vascular tissue.

16. The method of claim 11, wherein said dose of pharmaceutically active serelaxin ranges from about 10 μg/kg/day to about 500 μg/kg/day.

17. The method of claim 11, wherein said inflammation comprises an inflammatory disorder selected from the group consisting of eosinophilic airway hyperresponsiveness, asthma, rheumatoid arthritis, multiple sclerosis (MS), ankylosing spondylitis (AS), inflammatory bowel disease, gout, myositis, Sjogren's syndrome, systemic lupus erythematosus (SLE), vasculitis, pleural malignancy, sepsis, trauma, wound healing, atopic allergy, anaphylaxis, autoimmune encephalomyelitis, CNS hypoxia, CNS vascular damage, hypernociception eczema, dermatitis, scleroderma, poison ivy, acne, hives and psoriasis.

18. The method of claim 11, wherein said transient up-regulation of soluble ST-2 lasts from about 1 day to about 5 days.

19. The method of claim 1, wherein said soluble ST-2 binds Il-33.

20. The method of claim 19, wherein said soluble ST-2 functions as a decoy receptor lacking a transmembrane and/or cytoplasmic domain.

21. The method of claim 20, wherein said decoy receptor inhibits binding of said IL-33 to its transmembrane receptor ST-2.

22. The method of claim 17, wherein said inflammatory disorder is an IL-33-mediated immune response or an IL-33-mediated autoimmune disorder.
